(19) United States
(12) Patent Application Publication
(10) Pub. No.: US 2026/0123983 A1
(43) Pub. Date: May 7, 2026

(54) ACTUATION MECHANISMS FOR SURGICAL INSTRUMENTS AND SURGICAL INSTRUMENTS INCORPORATING THE SAME

(71) Applicant: Covidien LP, Mansfield, MA (US)

(72) Inventors: David D. Brause, Longmont, CO (US); Robert F. McCullough, JR., Boulder, CO (US); Monte S. Fry, Longmont, CO (US)

(73) Assignee: Covidien LP, Mansfield, MA (US)

(21) Appl. No.: 19/434,245

(22) Filed: Dec. 29, 2025

Related U.S. Application Data
(63) Continuation of application No. PCT/IB2024/056413, filed on Jul. 1, 2024.
(60) Provisional application No. 63/524,947, filed on Jul. 5, 2023.

(51) Int. Cl. A61B 18/14; A61B 34/35; A61B 17/00

(52) U.S. Cl. CPC A61B 18/1447; A61B 34/35; A61B 2017/00398; A61B 2018/1455

(57) ABSTRACT

A surgical instrument includes a shaft including a longitudinally extending slot, a drive element slidably disposed within the shaft, a drive link, and a body. The drive link includes a first portion external of the shaft and a second portion extending through the slot to engage the drive element within the shaft. The drive link defines a depth, a width greater than the depth, and a length. The depth of the drive link is less than the width of the slot to enable the drive link to extend through the slot. The width of the drive link is greater than the width of the slot. The body is disposed about at least a portion of the shaft. The first portion of the drive link is coupled to the body such that translation of the body along the shaft translates the drive element through the shaft.

### CROSS-REFERENCE TO RELATED APPLICATIONS

This application is a continuation of International Patent Application No. PCT/IB2024/056413, filed July 1, 2024, which claims the benefit of U.S. Provisional Patent Application Serial No. 63/524,947, filed July 5, 2023, which applications are incorporated herein by reference. To the extent appropriate, a claim of priority is made to each of the above disclosed applications.

### FIELD

The present disclosure relates to surgical instruments and, more particularly, to actuation mechanisms for surgical instruments such as, for example, to actuate a cutting element of a surgical instrument.

### BACKGROUND

Various different surgical instruments employ actuation mechanisms to actuate one or more operable components of the surgical instrument. A surgical forceps, for example, is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. Such electrosurgical forceps may include one or more actuation mechanisms to enable selective closure of the jaw members to grasp tissue for treating the grasped tissue and/or actuation of the knife to cut the treated tissue.

### SUMMARY

As used herein, the term “distal” refers to the portion that is being described which is farther from an operator (whether a human user (surgeon, nurse, etc.) or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any or all of the aspects detailed herein may be used in conjunction with any or all of the other aspects detailed herein.

Provided in accordance with the present disclosure is a surgical instrument including a shaft, a drive element, a drive link, and a body. The shaft includes a slot defined therein and extending longitudinally therealong. The slot defines a width. The drive element is slidably disposed within the shaft. The drive link includes a first portion positioned externally of the shaft and a second portion extending through the slot into an interior of the shaft to engage the drive element within the shaft. The drive link defines a depth, a width greater than the depth, and a length. The depth of the drive link is less than the width of the slot to enable the drive link to extend through the slot. The width of the drive link is greater than the width of the slot. The body is disposed about at least a portion of the shaft. The first portion of the drive link is coupled to the body such that translation of the body along the shaft translates the drive element through the shaft.

In an aspect of the present disclosure, the length of the drive link is greater than the width of the drive link.

In another aspect of the present disclosure, the second portion of the drive link is forked to define first and second spaced-apart prongs configured to engage the drive element therebetween.

In still another aspect of the present disclosure, the drive element includes a body and a leg disposed at an angle relative to the body. The drive link, in such aspects, is configured to engage the leg of the drive element.

In yet another aspect of the present disclosure, the drive link defines a plate configuration having opposed faces interconnected by an edge. In such aspects, the length and width of the drive link are defined across the opposed faces and the depth is defined across the edge.

In still yet another aspect of the present disclosure, at least one tab extends transversely from the first portion of the drive link.

In another aspect of the present disclosure, the first portion of the drive link is rotatably coupled to the body such the drive link is rotatable independently of the body.

Another surgical instrument provided in accordance with the present disclosure includes a housing, a shaft, an end effector assembly, a drive element, and a drive link. The shaft includes a proximal end portion disposed within the housing and a distal end portion at which the end effector assembly is disposed. The proximal end portion of the shaft includes a slot defined therein and extending longitudinally therealong. The slot defines a width. The drive element is slidably disposed within the shaft and configured to actuate a portion of the end effector assembly. The drive link is disposed within the housing and includes a first portion positioned externally of the shaft and a second portion extending through the slot into an interior of the shaft to engage the drive element within the shaft. The drive link defines a depth, a width greater than the depth, and a length. The depth of the drive link is less than the width of the slot to enable the drive link to extend through the slot. The width of the drive link is greater than the width of the slot. Translation of the drive link through the slot translates the drive element through the shaft to actuate the portion of the end effector assembly.

In an aspect of the present disclosure, the length of the drive link is greater than the width of the drive link.

In another aspect of the present disclosure, the second portion of the drive link is forked to define first and second spaced-apart prongs configured to engage the drive element therebetween.

In another aspect of the present disclosure, the drive element includes a body and a leg disposed at an angle relative to the body. The drive link is configured to engage the leg of the drive element.

In still another aspect of the present disclosure, the drive link defines a plate configuration having opposed faces interconnected by an edge. The length and width of the drive link are defined across the opposed faces and the depth is defined across the edge.

In yet another aspect of the present disclosure, a trigger is coupled to the housing and the drive link. Actuation of the trigger relative to the housing translates the drive link through the slot.

In still yet another aspect of the present disclosure, a body couples the trigger with the drive link. The first portion of the drive link is captured within the body.

Another surgical instrument provided in accordance with the present disclosure includes an outer shaft, a drive shaft slidably disposed within the outer shaft, an end effector assembly disposed at a distal end portion of the outer shaft, a drive element slidably disposed within the drive shaft, and a drive link. The drive shaft includes a slot defined therein and extending longitudinally therealong. The slot defines a width. The end effector assembly includes first and second jaw members, at least one of which is movable relative to the other to grasp tissue therebetween. The end effector assembly further includes a knife movable between the first and second jaw members to cut tissue grasped therebetween. The drive element is coupled to move the knife. The drive link extends through the slot into an interior of the drive shaft to engage the drive element within the drive shaft. The drive link defines a depth, a width greater than the depth, and a length. The depth of the drive link is less than the width of the slot to enable the drive link to extend through the slot. The width of the drive link is greater than the width of the slot. Translation of the drive link through the slot translates the drive element through the drive shaft to thereby move the knife relative to the first and second jaw members.

In an aspect of the present disclosure, the length of the drive link is greater than the width of the drive link.

In another aspect of the present disclosure, the outer shaft includes a slot and the drive link also extends through the slot of the outer shaft.

In still another aspect of the present disclosure, the drive shaft is slidable through the outer shaft to move the at least one of the first or second jaw members to grasp tissue therebetween. The drive shaft is configured to apply a force to the first and/or second jaw member of at least 80 lbs.

In yet another aspect of the present disclosure, the drive element includes a body and a leg disposed at an angle relative to the body. The drive link is configured to engage the leg of the drive element.

In still yet another aspect of the present disclosure, the drive link includes a forked portion having first and second spaced-apart prongs configured to engage the drive element therebetween.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a side view of an electrosurgical system in accordance with the present disclosure including an electrosurgical forceps and an electrosurgical generator;

FIG. 2 is a side, cutaway view of a proximal portion of the electrosurgical forceps of the system of FIG. 1;

FIG. 3 is a top, longitudinal, cross-sectional view taken along section line 3-3 of FIG. 2;

FIG. 4 is a perspective view of an end effector assembly of the electrosurgical forceps with first and second jaw members of the end effector assembly disposed in a spaced-apart configuration;

FIG. 5 is a side view of the end effector assembly of the electrosurgical forceps with the first and second jaw members moving from the spaced-apart position configuration towards an approximated position for grasping tissue therebetween;

FIG. 6 is a side view of the end effector assembly of the electrosurgical forceps with the first and second jaw members disposed in the approximated configuration grasping tissue therebetween;

FIG. 7 is a side view of the end effector assembly of the electrosurgical forceps with the first and second jaw members disposed in the approximated position and a knife advanced between the first and second jaw members to cut the grasped tissue;

FIG. 8 is a side, cutaway view of the proximal portion of the electrosurgical forceps with a drive assembly and a trigger assembly of the electrosurgical forceps actuated, wherein internal portion of the electrosurgical forceps are removed to better illustrate operation of the drive and trigger assemblies;

FIG. 9 is an enlarged, side view of a portion of the trigger assembly operably disposed about an outer shaft of the electrosurgical forceps;

FIG. 10 is a side, longitudinal, cross-sectional view of a spindle and drive link of the trigger assembly operably coupled to the outer shaft, a drive shaft, and a knife rod of the electrosurgical forceps;

FIG. 11 is a transverse, cross-sectional view of the drive link of the trigger assembly operably coupled to the outer shaft, the drive shaft, and the knife rod of the electrosurgical forceps;

FIG. 12 is a top view of the drive link of the trigger assembly operably coupled to the outer shaft, the drive shaft, and the knife rod of the electrosurgical forceps;

FIG. 13 is a longitudinal, cross-sectional view of the drive link of the trigger assembly operably coupled to the outer shaft, the drive shaft, and the knife rod of the electrosurgical forceps;

FIG. 14 is a perspective view of the drive link of the trigger assembly;

FIGS. 15 and 16 are perspective and exploded, perspective views of the spindle of the trigger assembly;

FIG. 17 is a perspective view of another drive link configured for use with the trigger assembly;

FIG. 18 is a transverse, cross-sectional view of the drive link of FIG. 17 engaged within the spindle of the trigger assembly and operably coupled to the outer shaft, the drive shaft, and the knife rod of the electrosurgical forceps;

FIGS. 19-23 are perspective views of other drive links configured for use with the trigger assembly;

FIG. 24 is a perspective view of the drive link of FIG. 23 prior to assembly of the tab thereon; and

FIG. 25 is a schematic illustration of a robotic surgical system configured for use in accordance with aspects of the present disclosure.

### DETAILED DESCRIPTION

The present disclosure provides actuation mechanisms for surgical instruments such as, for example, to actuate a knife of an electrosurgical forceps. Although described herein with respect to actuating a knife of an electrosurgical forceps, the actuation mechanisms of the present disclosure are equally applicable for use with any other suitable surgical instruments.

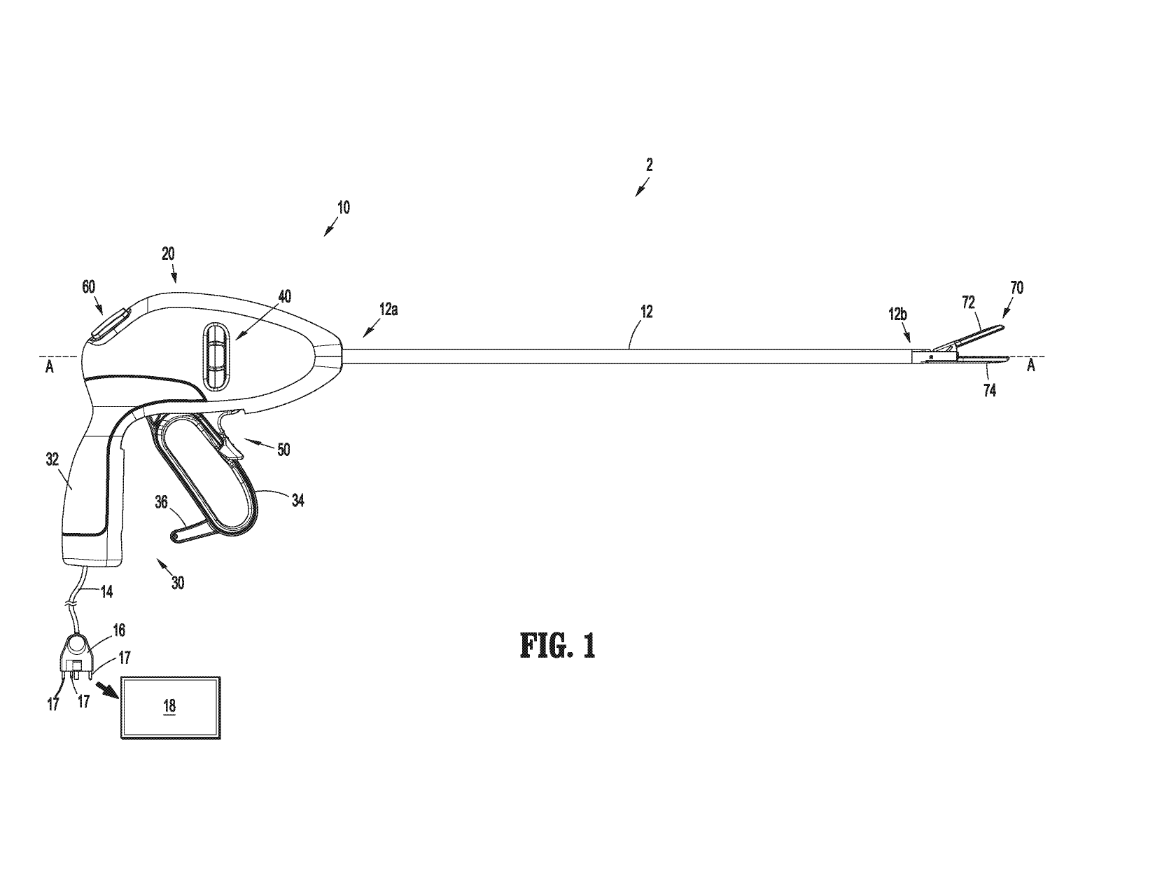

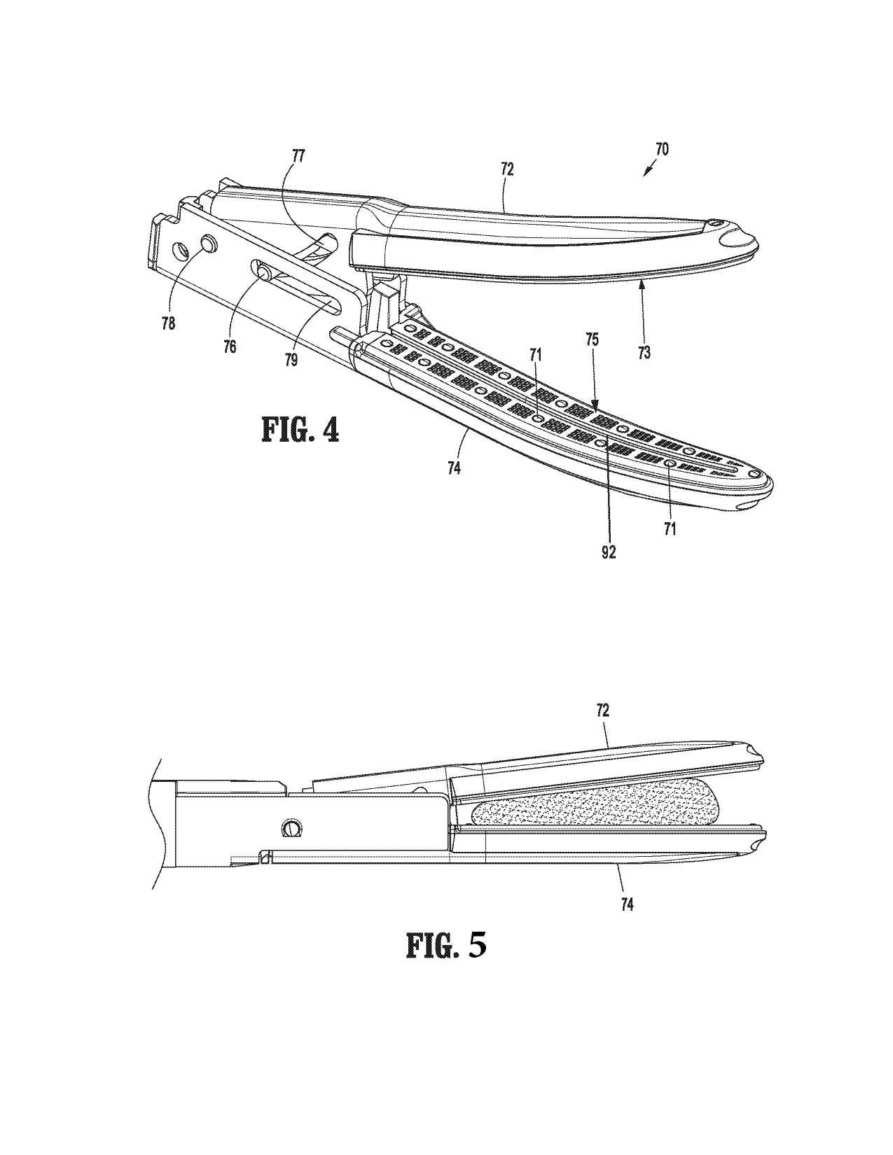

Referring to FIG. 1, an electrosurgical system 2 provided in accordance with the present disclosure includes an electrosurgical forceps 10 and an electrosurgical generator 18. Forceps 10 includes a housing 20, a handle assembly 30, a rotating assembly 40, a trigger assembly 50, an activation assembly 60, and an end effector assembly 70. End effector assembly 70 includes first and second jaw members 72, 74, at least one of which is pivotable relative to the other about a pivot pin 78 (FIG. 4; or other suitable pivot structure) to grasp tissue to enable sealing and/or dividing of the grasped tissue. End effector assembly 70 further includes a knife 90 (FIGS. 7 and 18) operably coupled to trigger assembly 50 to enable selective translation of knife 90 (FIG. 7) between jaw members 72, 74 to cut tissue (e.g., previously sealed tissue) grasped between jaw members 72, 74, as described in greater detailed below.

Forceps 10 further includes an outer shaft 12 that defines a longitudinal axis “A-A” and has a proximal end portion 12a operatively engaged to housing 20 and a distal end portion 12b operably engaged to end effector assembly 70. Forceps 10 also includes an electrosurgical cable 14 having a plug 16 configured to connect forceps 10 to generator 18. More specifically, plug 16 includes a plurality of pins 17 or other suitable contacts configured to mate with corresponding receptacles or other contacts of a port (not shown) of generator 18 to enable generator 18 to communicate with forceps 10 and control the supply of electrosurgical energy to end effector assembly 70 of forceps 10 for sealing tissue grasped between first and second jaw members 72, 74.

Referring to FIGS. 1-3, handle assembly 30 includes a fixed handle 32 and a movable handle 34. Fixed handle 32 is integrally associated with housing 20 and movable handle 34 is movable relative to the fixed handle 32 to actuate a drive assembly 80 of forceps 10. Movable handle 34 has an upper end portion 34a that is pivotally secured within housing 20 and operably engaged with drive assembly 80. Drive assembly 80 includes a plurality of linkages 82, a carriage 84, a compression spring 86, and a drive shaft 88. Upper end portion 34a of movable handle 34 operably couples to linkages 82 that cooperate to move carriage 84 distally against compression spring 86 in response to actuation of movable handle 34 towards fixed handle 32. Movement of carriage 84 distally against compression spring 86, in turn, regulates translation of drive shaft 88 through outer shaft 12 and relative to end effector assembly 70 to move either or both of jaw members 72, 74 relative to one another to grasp tissue between jaw members 72, 74 and regulate the closure force applied to tissue grasped between jaw members 72, 74.

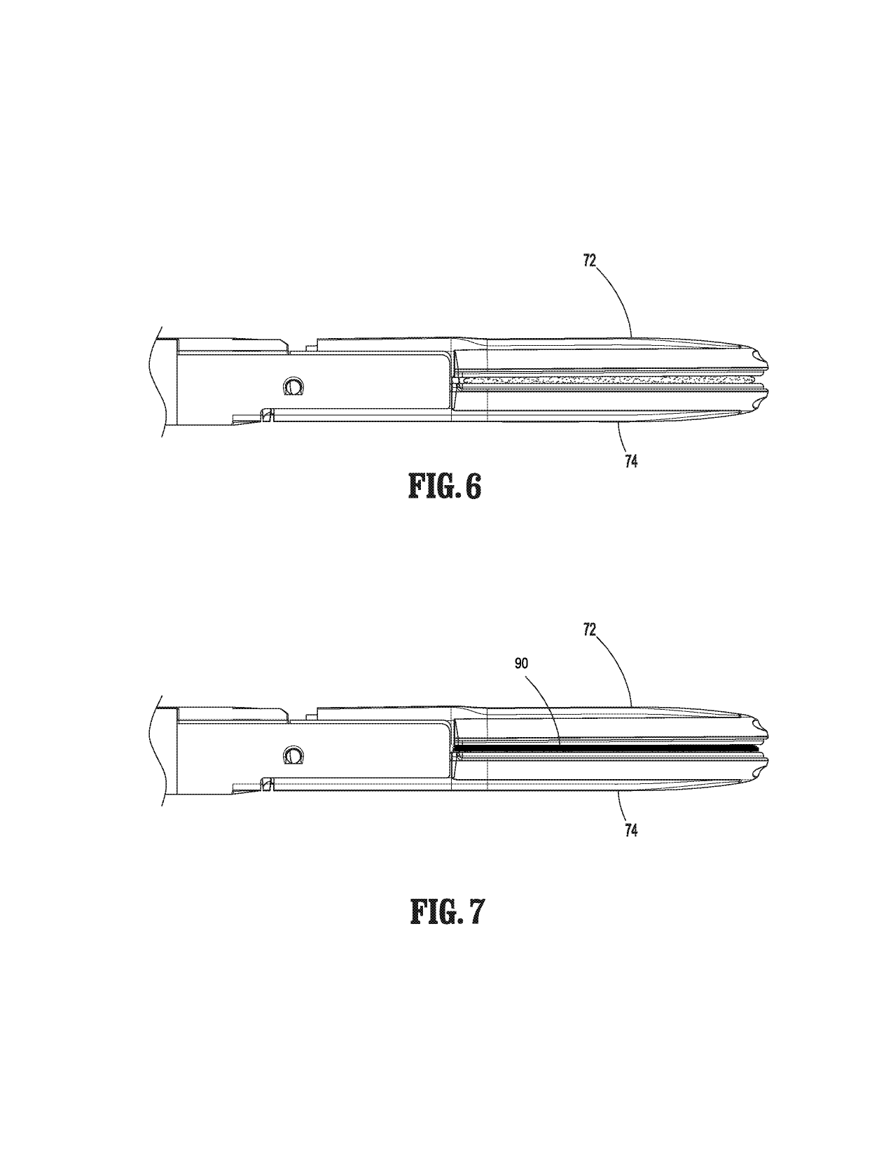

With additional reference to FIGS. 4-6, movement of carriage 84 distally against compression spring 86 initially urges compression spring 86 to translate distally to thereby translate drive shaft 88 distally through outer shaft 12 to drive relative movement of cam pin 76 through cam slots 77, 79 defined within respective first and second jaw members 72, 74 to thereby urge jaw member 72 to pivot about pivot pin 78 and relative to jaw member 74 from a spaced-apart configuration (FIG. 4) through an intermediate position (FIG. 5) towards an approximated configuration (FIG. 6) to grasp tissue between first and second jaw members 72, 74 and apply a closure force to the grasped tissue. As an alternative to distal movement of drive shaft 88 closing jaw members 72, 74, it is also contemplated that the opposite configuration be provided, e.g., wherein proximal movement of drive shaft 88 closes jaw members 72, 74, or that other suitable closure mechanisms be provided, e.g., wherein rotation of a drive element closes jaw members 72, 74.

Upon reaching a threshold closure force applied to tissue grasped between first and second jaw members 72, 74, further movement of carriage 84 distally against compression spring 86, e.g., in response to further actuation of movable handle 34 towards fixed handle 32, compresses compression spring 86 rather than translating compression spring 86 distally (due to the resistive force applied by tissue inhibiting further closure of jaw members 72, 74) such that drive shaft 88 and, thus, first and second jaw members 72, 74 are maintained in position grasping tissue therebetween. In this manner, the closure force applied to tissue grasped between jaw members 72, 74 is regulated to maintain a closure force or closure force within a closure force range.

In aspects, the closure force applied to the grasped tissue in the approximated configuration of jaw members 72, 74 may be regulated such that the closure pressure, measured at a jaw centroid along the lengths of jaw members 72, 74, may be in a range of (or the jaw pressure range may be) from about 70 psi to about 130 psi; in other aspects from about 80 psi to about 120 psi; and, in still other aspects, from about 90 psi to about 110 psi.

In order to achieve the above-noted closure forces or closure force ranges (or to achieve other suitable closure forces or closure force ranges), a suitable force must be applied by drive shaft 88 to either or both of jaw members 72, 74 to urge jaw members 72, 74 towards the approximated configuration grasping tissue therebetween under the closure pressure or closure pressure within the closure pressure range. This force applied by drive shaft 88 may be, in aspects, at least about 80 lbs., in other aspects, at least about 120 lbs. and, in still other aspects, at least about 160 lbs. Thus, drive shaft 88 must have sufficient strength to withstand these forces during use.

Continuing with reference to FIGS. 1-6, the materials and configuration of drive shaft 88 at least partially define the strength of drive shaft 88. More specifically, with respect to the configuration of drive shaft 88, the outer diameter, inner diameter (of the internal lumen), thickness, and any features defined within drive shaft 88 impact the strength of drive shaft 88. For example, slots, apertures, cut-outs, etc. defined within drive shaft 88 serve to decrease the overall strength of drive shaft 88. Thus, the strength of drive shaft 88 may be preserved by minimizing or eliminating such features defined within drive shaft 88 (e.g., minimizing the width of any slots, minimizing the diameter of any apertures, etc.).

End effector assembly 70 is described as a unilateral assembly, e.g., wherein second jaw member 74 is fixed relative to outer shaft 12 and first jaw member 72 is pivotable relative to second jaw member 74 and outer shaft 12; however, a bilateral assembly, e.g., wherein both first and second jaw members 72, 74 are pivotable relative to one another and outer shaft 12, is also contemplated. Further, in order to drive relative movement of cam pin 76 through cam slots 77, 79, drive shaft 88 may be translationally fixed to cam pin 76 and outer shaft 12 translationally fixed to pivot pin 78 such that translation of drive shaft 88 moves cam pin 76 relative to jaw members 72, 74 (and, thus, cam slots 77, 79) to thereby drive relative movement of cam pin 76 through cam slots 77, 79 to pivot first jaw member 72 towards second jaw member 74 to grasp tissue therebetween and apply a closure force to the grasped tissue. Alternatively, drive shaft 88 may be translationally fixed to pivot pin 78 and outer shaft 12 translationally fixed to cam pin 76 such that translation of drive shaft 88 moves jaw members 72, 74 (and, thus, cam slots 77, 79) relative to cam pin 76 to thereby drive relative movement of cam pin 76 through cam slots 77, 79 to pivot first jaw member 72 towards second jaw member 74 from the spaced-apart configuration (FIG. 4) towards the approximated configuration (FIG. 6) to grasp tissue between first and second jaw members 72, 74 and apply the closure force to the grasped tissue.

With reference to FIG. 4, each jaw member 72, 74 of end effector assembly 70 includes an electrically conductive tissue contacting surface 73, 75, respectively. Jaw members 72, 74 are configured to grasp tissue between electrically conductive tissue contacting surface 73, 75 in the approximated configuration thereof. Electrically conductive tissue contacting surfaces 73, 75 are adapted to connect to generator 18 (FIG. 1), e.g., via suitable electrical lead wires, electrically conductive structures, or combinations thereof extending through outer shaft 12, housing 20, and electrosurgical cable 14 to corresponding pins 17 of plug 16 (FIG. 1), to enable energization of electrically conductive tissue contacting surface 73, 75 with electrosurgical, e.g., Radio Frequency (RF), energy at different potentials for conducting electrosurgical energy between electrically conductive tissue contacting surface 73, 75 and through tissue grasped therebetween to seal the tissue.

Either or both jaw members 72, 74 may further include one or more stop members 71 (FIG. 4) disposed on or otherwise associated with either or both tissue-contacting surfaces 73, 75 to maintain a minimum gap distance between tissue contacting surfaces 73, 75 (or to maintain a gap distance within a gap distance range between tissue contacting surfaces 73, 75) when jaw members 72, 74 are disposed in a fully approximated position, thus inhibiting electrical shorting. Stop member(s) 71 may be insulative, partly insulative, and/or electrically isolated from either or both tissue contacting surfaces 73, 75. In aspects, the minimum gap distance or the gap distance range may be from about 0.001 inches to about 0.010 inches; in other aspects from about 0.001 inches to about 0.008 inches; and, in still other aspects from about 0.001 inches to about 0.006 inches. Other suitable gap distances and ranges are also contemplated. The gap distance may be determined as the maximum gap distance between the tissue contacting surfaces 73, 75.

Either or both jaw members 72, 74 may additionally or alternatively include a longitudinally extending knife channel 92 defined therethrough. Knife channel(s) 92 is configured to receive a portion of knife 90 (FIG. 7) as knife 90 (FIG. 7) is translated between a retracted configuration, wherein knife 90 (FIG. 7) is disposed proximally of tissue contacting surfaces 73, 75, and an extended configuration (see FIG. 7), wherein knife 90 (FIG. 7) extends between tissue contacting surfaces 73, 75 to cut tissue grasped therebetween. Knife 90 (FIG. 7) is operably coupled to trigger assembly 50 (FIGS. 1-3) which, as detailed below, is configured to enable the selective actuation of knife 90 (FIG. 7) between the retracted and extended configurations.

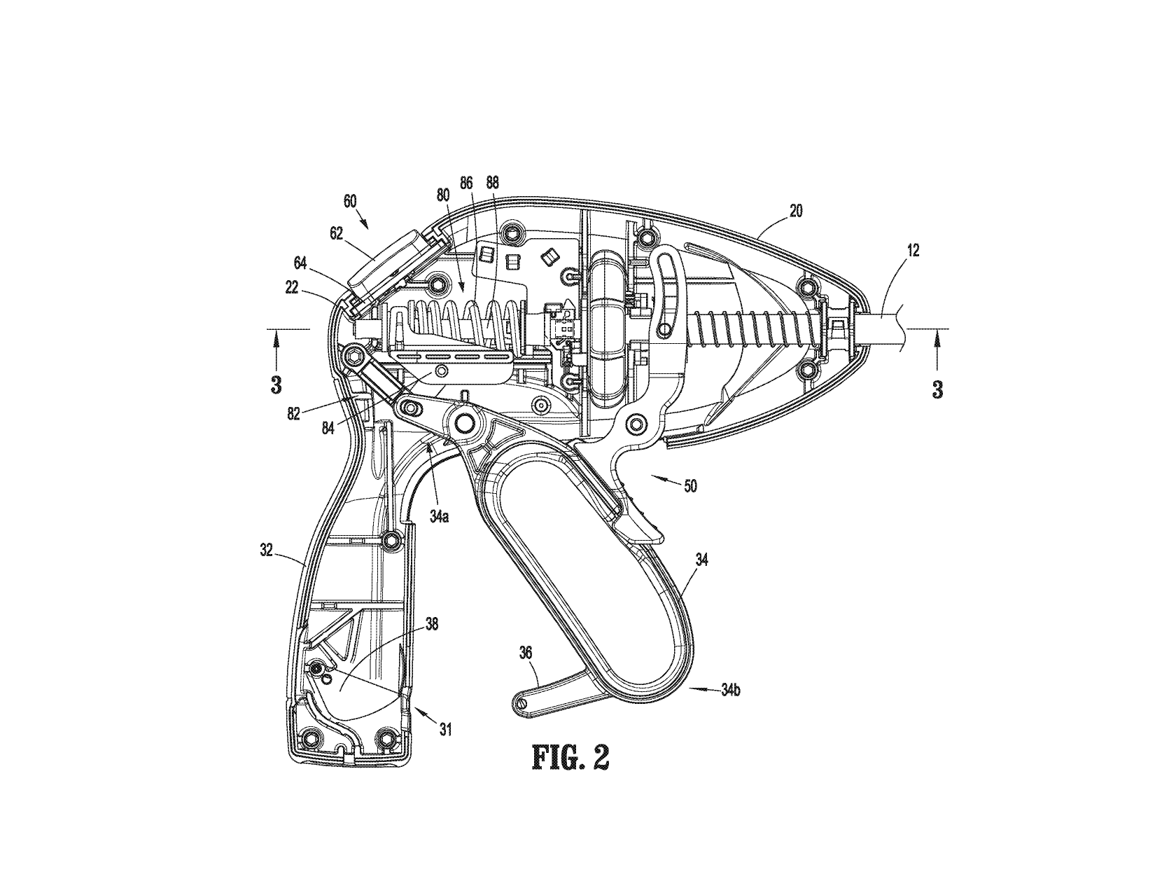

Referring back to FIGS. 1 and 2, movable handle 34 includes a flange 36 extending proximally from a lower end portion 34b of movable handle 34. Flange 36 is configured to extend through an aperture 31 defined within fixed handle 32 and ultimately engage a latch 38 within fixed handle 32 that is configured to selectively lock and unlock the fixed and movable handles 32, 34 relative to one another upon sufficient actuation of movable handle 34. Upon initial movement of flange 36 through aperture 31 to engage latch 38, in response to an initial actuation of movable handle 34 towards fixed handle 32, fixed and movable handles 32, 34 are locked relative to one another to thereby latch first and second jaw members 72, 74 in the approximated configuration. Upon subsequent movement of flange 36 within aperture 31, in response to a subsequent actuation of movable handle 34 towards fixed handle 32, flange 36 is disengaged from latch 38 such that fixed and movable handles 32, 34 are unlocked permitting return of movable handle 34 towards its initial position and return of jaw members 72, 74 towards the spaced-apart configuration. In aspects, flange 36 and latch 38 are omitted and movable handle 34 is manually maintained in approximation with fixed handle 32 to thereby maintain first and second jaw members 72, 74 in the approximated configuration.

Rotating assembly 40 is engaged with outer shaft 12 within housing 20 and extends outwardly from either side of housing 20 to enable a user to manually control the orientation of outer shaft 12 and thus, end effector assembly 70, relative to housing 20. In aspects, rotating assembly 40 is infinitely rotatable in either direction about the longitudinal axis “A-A” to similarly rotate end effector assembly 70 relative to housing 20. Alternatively, rotating assembly 40 may have a defined range of motion.

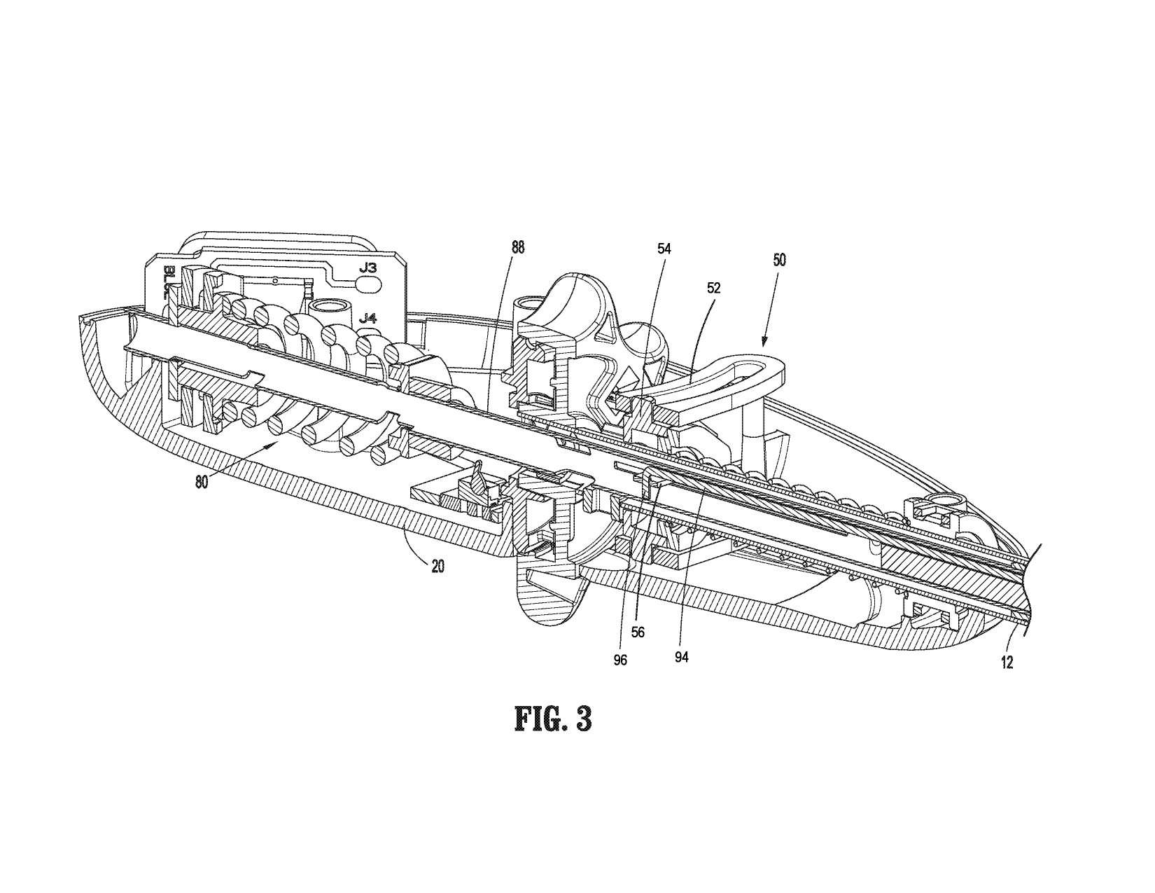

With additional reference to FIG. 3, activation assembly 60 is configured to signal generator 18 to initiate the supply of electrosurgical energy to first and second jaw members 72, 74 for sealing tissue. Activation assembly 60 includes an activation button 62 supported by a body 22 of housing 20. Activation button 62 is movable between an un-actuated position and an actuated position to thereby transition an underlying electrical switch 64 between a first state and a second state. Electrical switch 64, in turn, is adapted to electrically connect to generator 18, e.g., via one or more electrical lead wires extending from electrical switch 64 through housing 20 and electrosurgical cable 14 to one of the pins 17 (or other contact) of plug 16 to enable communication of the state of the electrical switch to generator 18. Generator 18, more specifically, may be configured to read an output of a corresponding pin 17, e.g., the presence of a resistance, voltage, current, etc. and/or a value of the resistance, voltage, current, etc., to detect the state of electrical switch 64 and, thus, to detect whether the user has activated activation button 62. For example, generator 18 may read the first state of electrical switch 64 as corresponding to an un-activated state and the second state of electrical switch 64 as corresponding to an activated state.

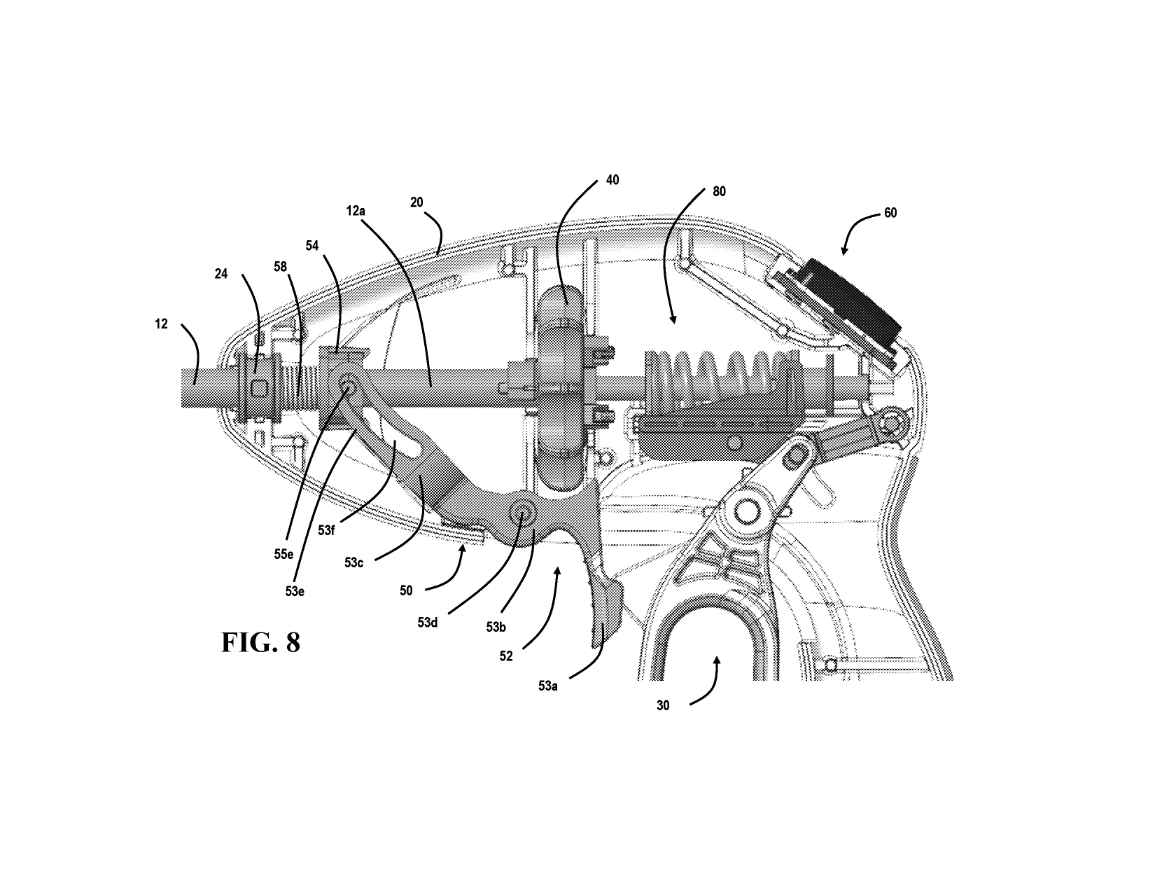

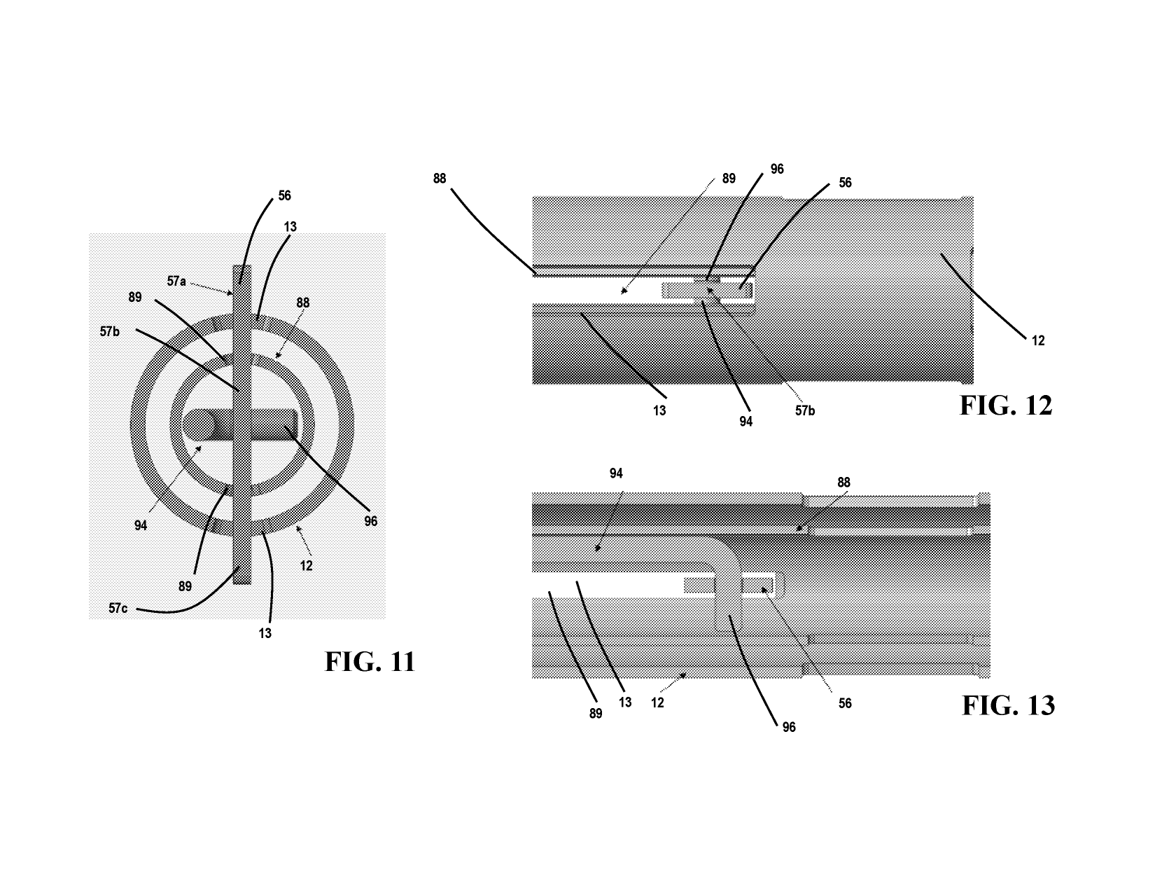

With reference to FIGS. 2-4 and 8-16, trigger assembly 50 enables selective actuation of knife 90 (FIGS. 7 and 18) relative to jaw members 72, 74 to cut tissue grasped between jaw members 72, 74, e.g., once the grasped tissue is sealed. Knife 90 (FIG. 7) may be configured for mechanically cutting tissue or electromechanically cutting tissue wherein knife 90 (FIG. 7) is energized (e.g., with electrosurgical energy) to facilitate tissue cutting. Trigger assembly 50 includes a trigger 52, a spindle 54, a drive link 56, and a biasing spring 58. Trigger assembly 50 is coupled to knife drive rod 94 which, in turn, is engaged to knife 90 (FIGS. 7 and 18), either directly or via one or more intermediate components. In aspects, knife 90 (FIG. 7) is welded to a distal end portion of knife drive rod 94 (see FIG. 18). Regardless of the manner of connection, actuation of trigger 52 of trigger assembly 50 from an un-actuated configuration (FIG. 4) to an actuated configuration (FIG. 8) deploys knife 90 (FIGS. 7 and 18) relative to first and second jaw members 72, 74 from the retracted configuration to the extended configuration.

Trigger 52 includes a manipulation portion 53a, a body portion 53b, and an actuation portion 53c. Body portion 53b of trigger 52 is pivotably engaged to housing 20 within housing 20, e.g., via pivotable engagement of opposing pivot bosses 53d of body portion 53b of trigger 52 within corresponding pivot recesses (not shown) defined on opposing interior surfaces of housing 20. Manipulation portion 53a of trigger 52 extends from body portion 53b and through an opening in housing 20 such that at least a portion of manipulation portion 53a is exposed exteriorly of housing 20 to enable manual manipulation of trigger 52 by a user. Actuation portion 53c extends from body portion 53b further into housing 20, e.g., substantially opposite manipulation portion 53a, such that body portion 53b is disposed between manipulation portion 53a and actuation portion 53c. Actuation portion 53c defines a bifurcated configuration including a pair of spaced-apart flanges 53e each defining a cam slot 53f.

Spindle 54 includes a body 55a defining a substantially cylindrical interior cavity 55b. In aspects, body 55a is formed from a base 55c and a cap 55d configured to engage one another, e.g., in snap-fit engagement, to form body 55a. Body 55a further includes a cam boss 55e protruding outwardly from opposing sides of body 55a. Spindle 54 is configured for positioning between flanges 53e of actuation portion 53c of trigger 52 with cam bosses 55e extending into corresponding cam slots 53f. Further, spindle 54 is configured for slidable positioning about proximal end portion 12a of outer shaft 12 within housing 20.

Drive link 56 is rotatably captured within interior cavity 55b of body 55a of spindle 54 such that drive link 56 is longitudinally fixed but rotatable within and relative to spindle 54. Drive link 56, as described in greater detail below, is engaged with knife drive rod 94 within spindle 54.

Actuation of trigger 52 of trigger assembly 50 from the un-actuated configuration (FIG. 4) to the actuated configuration (FIG. 8) is accomplished by pulling manipulation portion 53a of trigger 52 proximally towards handle assembly 30. As a result of this proximal movement of manipulation portion 53a, trigger 52 is pivoted relative to housing 20 such that actuation portion 53c of trigger 52 is moved distally within housing 20. This distal movement of actuation portion 53c urges spindle 54 distally along outer shaft 12 as cam bosses 55e of spindle 54 move through cam slots 53e of actuation portion 53c of trigger 52. The distal sliding of spindle 54 along outer shaft 12 likewise slides drive link 56 distally relative to outer shaft 12 (e.g., through opposing longitudinal slots 13 defined within outer shaft 12) to, in turn, move knife drive rod 94 through outer shaft 12. Since knife 90 (FIG. 7) is engaged with knife drive rod 94, this distal movement of knife drive rod 94 through outer shaft 12 advances knife 90 (FIG. 7) relative to first and second jaw members 72, 74 from the retracted configuration to the extended configuration.

Biasing spring 58 is disposed about outer shaft 12 within housing 20 between spindle 54 and a collar 24 (FIG. 8) supported within housing 20. Biasing spring 58 is positioned to bias spindle 54 proximally within housing 20 and relative to outer shaft 12, thereby biasing drive link 56 and knife drive rod 94 proximally such that knife 90 (FIG. 7) is biased towards the retracted configuration. Further, this bias provided by biasing spring 58 biases trigger 52 towards the un-actuated configuration.

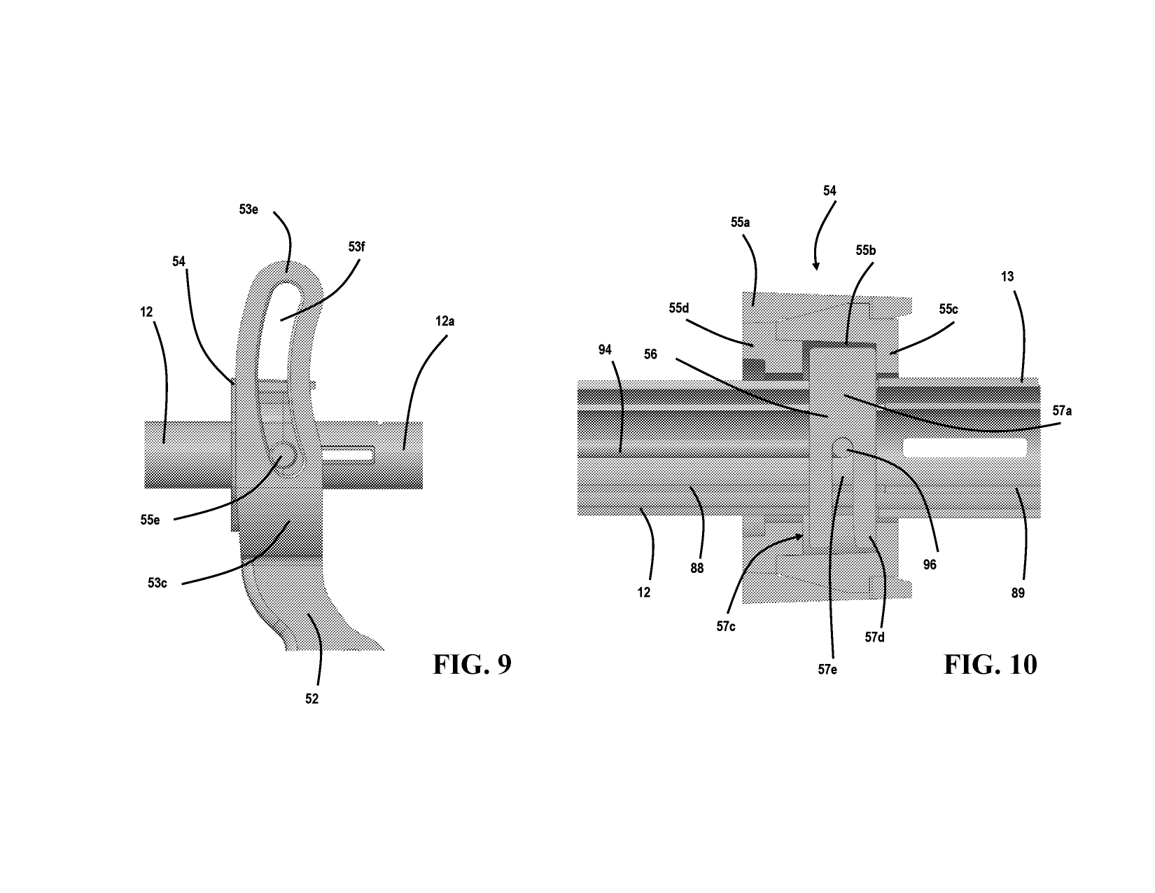

Referring to FIGS. 9-16, as detailed above, spindle 54 is slidably disposed about outer shaft 12 and drive shaft 88 is slidably disposed within outer shaft 12. Further, knife drive rod 94 is slidably disposed within outer shaft 12. Thus, in order for drive link 56 to engage knife drive rod 94 with spindle 54 such that actuation of trigger 52 moves knife drive rod 94, drive link 56 extends through a pair of first transversely opposed, longitudinally extending slots 13 defined within outer shaft 12 and a pair of second transversely opposed, longitudinally extending slots 89 defined within drive shaft 88. Drive link 56, more specifically, is configured to provide the requisite coupling of knife drive rod 94 with spindle 54 while enabling minimization of widths of second slots 89 defined within drive shaft 88, thus minimizing any loss of strength of drive shaft 88 such that drive shaft 88 is capable of imparting suitable forces to jaw members 72, 74 to achieve a suitable jaw closure force or jaw closure force within a suitable range without failing, e.g., buckling.

Drive link 56 defines a plate configuration having opposed broad faces 57a (defining the width and length dimensions of drive link 56) interconnected by narrow edges 57b (defining the depth dimension of drive link 56). Drive link 56 defines a rectangular cross-sectional configuration wherein the width dimension is greater than the depth dimension (and the length dimension is greater than the width dimension). In aspects, the width dimension is at least about 50% greater than the depth dimension; in other aspects, the width dimension is at least about 75% greater than the depth dimension; in still other aspect, the width dimension is at least about 100% greater than the depth dimension. Other configurations are also contemplated. Drive link 56 may define substantially uniform depth, width, and/or height dimensions and/or may define varied depth, width, and/or height dimensions. In configurations wherein one or more dimensions is varied, the dimensions noted herein may correspond to the portions of drive link 56 that extend into outer shaft 12 (and drive shaft 88) and/or may be average dimensions.

Drive link 56 may be formed from a metal, a plastic, may include metal parts and plastic parts, or may be formed from any other suitable material(s) or combinations of materials. In aspects, drive link 56 is formed via stamping, injection molding, overmolding, laser cutting, combinations thereof, and/or any other suitable manufacturing process.

Drive link 56 includes a forked portion 57c including first and second spaced-apart prongs 57d extending in the length dimension of drive link 56 and defining a slot 57e disposed between prongs 57d and extending through opposed broad faces 57a of drive link 56. Prongs 57d may extend, in aspects, about 30% to about 70% of the length dimension of drive link 56; in other aspects, prongs 57d may extend about 40% to about 60% of the length dimension of drive link 56.

Continuing with reference to FIGS. 9-16, prongs 57d are configured to capture a proximal leg 96 of knife drive rod 94 within slot 57e to engage drive link 56 with knife drive rod 94 such that translation of drive link 56, e.g., in response to translation of spindle 54, similarly translates knife drive rod 94. Proximal leg 96 extends from the longitudinally-extending body of knife drive rod 94. In aspects, proximal leg 96 (or at least the portion thereof that is engaged by drive link 56) is angled, e.g., bent, relative to the longitudinally-extending body of knife drive rod 94 at an angle of between about 60 degrees and about 120 degrees; in other aspects, from about 75 degrees to about 105 degrees; and, in other aspects, about 90 degrees. In this manner, with drive link 56 engaged with proximal leg 96 of knife drive rod 94, drive link 56 is oriented with the width dimension thereof extending longitudinally and the depth dimension thereof extending transversely. In aspects, prongs 57d define tapered inner surfaces 57f at the free ends thereof to define an enlarged open end of slot 57e, thus facilitating moving drive link 56 into engagement about proximal leg 96 of knife drive rod 94.

Prongs 57d of drive link 56 are engaged with proximal leg 96 of knife drive rod 94 within drive shaft 88. More specifically, drive link 56 is inserted, led by prongs 57d, through one of the first slots 13 of outer shaft 12, one of the second slots 89 of drive shaft 88, about proximal leg 96 to engage proximal leg 96 within slot 57e, through the other second slot 89 of drive shaft 88, and through the other first slot 13 of outer shaft 12. Thus, with drive link 56 engaged about proximal leg 96, drive link 56, in the length dimension thereof, extends through and protrudes from opposing sides of outer shaft 12 (and, thus, drive shaft 88, which is disposed within (e.g., coaxially within) outer shaft 12).

The plate (e.g., rectangular) configuration of drive link 56 enables drive link 56 to have a width dimension that provides suitable strength to drive link 56 while also enabling the depth of drive link 56 to be relatively small. That is, with a plate (e.g., rectangular) configuration, strength and support can be provided to drive link 56 by varying the width dimension without requiring an increase, or requiring a relatively small increase, in the depth dimension. The relatively small depth dimension of drive link 56 enables slots 89 (and slots 13) to define relatively minimal widths such that, as noted above, any loss of strength of drive shaft 88 due to the presence of slots is minimized. More specifically, while the widths of slots 89 are required to be greater than the depth of drive link 56 (to enable receipt of drive link 56 therethrough), the plate configuration of drive link 56 enables the width dimension of drive link 56 to be greater than the width of slots 89, thus adding strength to drive link 56 without requiring enlargement of the widths of slots 89. This contrasts with a cylindrical drive link such as a pin, wherein increasing the diameter necessarily increases both thickness and width dimensions of the pin, thereby requiring an increase in the widths of the slot(s) through which the pin extends.

In aspects, the width dimension of drive link 56 is about 30% greater than a width of slots 89; in other aspects, about 40% greater than the width of slots 89; in still other aspects, about 50% greater than the width of slots 89. Again, as noted above, a pin drive link cannot have a width dimension (or any diameter) greater than the width of slots 89.

The portions of drive link 56 that protrude from the opposing sides of outer shaft 12 are captured within interior cavity 55b of body 55a of spindle 54, e.g., via engagement of base 55c and cap 55d with one another with drive link 56 disposed therebetween. Thus, sliding of spindle 54 along outer shaft 12, e.g., in response to actuation of trigger 52, likewise slides drive link 56 and, thus, knife drive rod 94, relative to outer shaft 12 and drive shaft 88. However, drive link 56 is rotatably captured within interior cavity 55b of body 55a of spindle 54 such that, in response to rotation of rotating assembly 40 relative to housing 20, outer shaft 12, drive shaft 88, end effector assembly 70 (including knife 90 (FIG. 7)), knife drive rod 94, and drive link 56 are likewise rotated relative to housing 20 while spindle 54 and the remainder of trigger assembly 50 are not rotated.

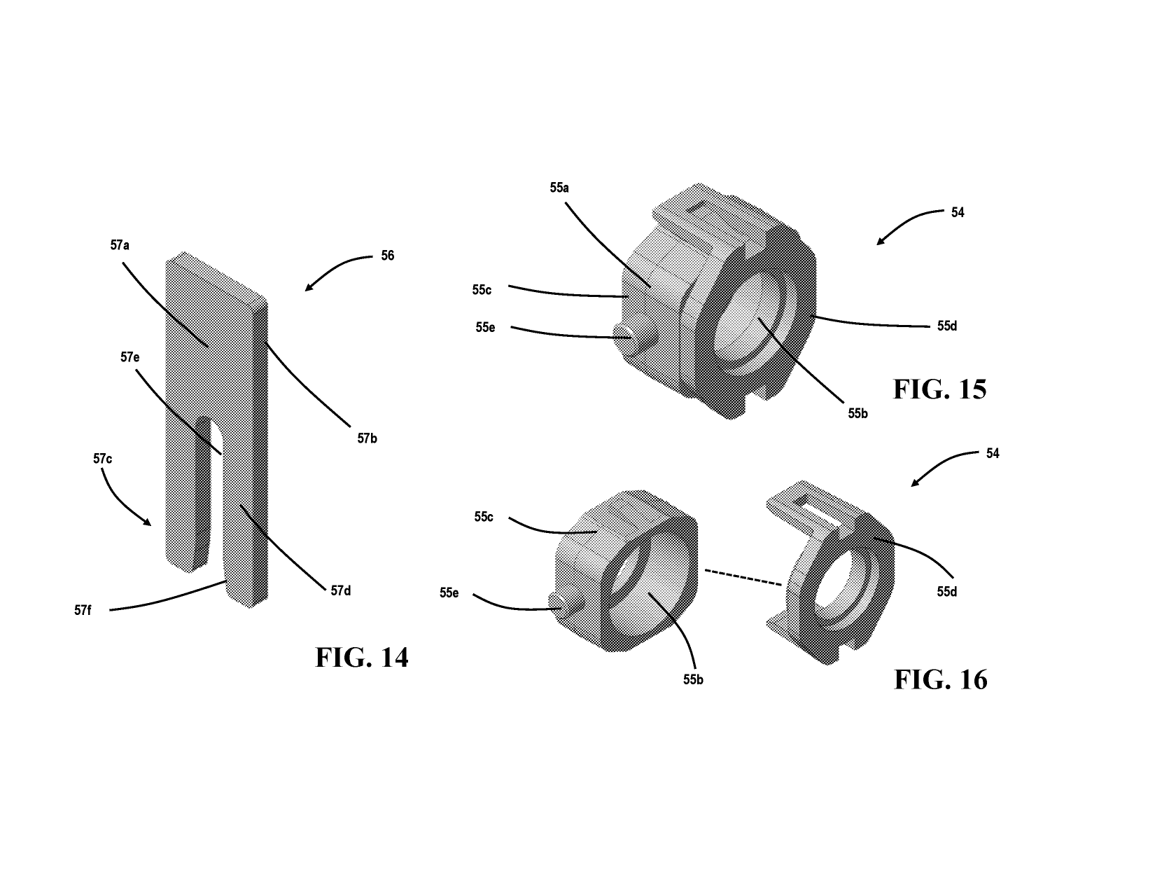

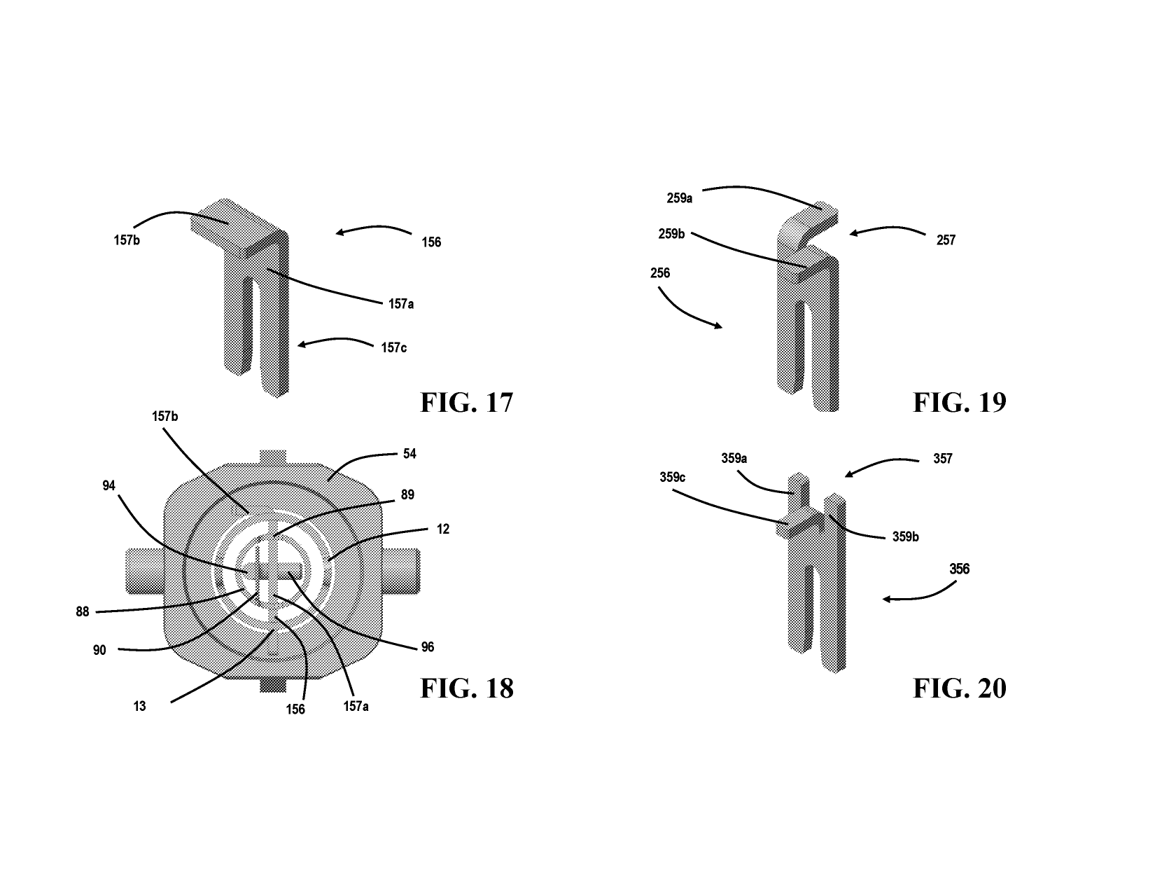

Turning to FIGS. 17 and 18, another drive link 156 provided in accordance with the present disclosure is shown. Drive link 156 is similar to and may include any of the features of drive link 56 (FIG. 14) except as explicitly contradicted below. Accordingly, similarities between drive link 156 and drive link 56 (FIG. 14) are summarily described or omitted hereinbelow for purposes of brevity.

Drive link 156 includes a plate body 157a having a folded tab 157b disposed at one end thereof and a forked portion 157c disposed at the opposite end thereof. Folded tab 157b may extend at an angle relative to a plane defined by plate body 157a of, in aspects, about 75 degrees to about 105 degrees; or, in other aspects, at an angle of about 90 degrees. With drive link 156 extending through outer shaft 12, drive shaft 88, and engaging knife drive rod 94, one of the portions of drive link 156 that protrudes from outer shaft 12 includes folded tab 157b, e.g., such that folded tab 157b remains external of outer shaft 12. Folded tab 157b is configured to inhibit passage through slot 13 of outer shaft 12, thus maintaining folded tab 157b external of outer shaft 12. Folded tab 157b, in addition to the free end of forked portion 157c, is rotatably captured within spindle 54, similarity as detailed above with respect to drive link 56 (FIG. 14). Drive link 156 may be formed from metal via stamping or in any other suitable manner and/or using any other suitable material(s).

FIGS. 19-21 illustrate additional drive links 256, 356, 456 including folded tabs 257, 357, 457, respectively. Drive links 256, 356, 456 differ from drive link 156 (FIG. 14) only in the configuration of folded tabs 257, 357, 457, respectively, and, thus, similar features are summarily described or omitted hereinbelow for purposes of brevity.

Referring to FIG. 19, folded tab 257 of drive link 256 includes first and second fingers 259a, 259b extending in opposite directions and at an angles of, in aspects, about 75 degrees to about 105 degrees; or, in other aspects, about 90 degrees.

With reference to FIG. 20, folded tab 357 of drive link 356 includes first and second outer uprights 359a, 359b and an inner finger 359c disposed between outer uprights 359a, 359b and extending at an angle of, in aspects, about 75 degrees to about 105 degrees; or, in other aspects, about 90 degrees.

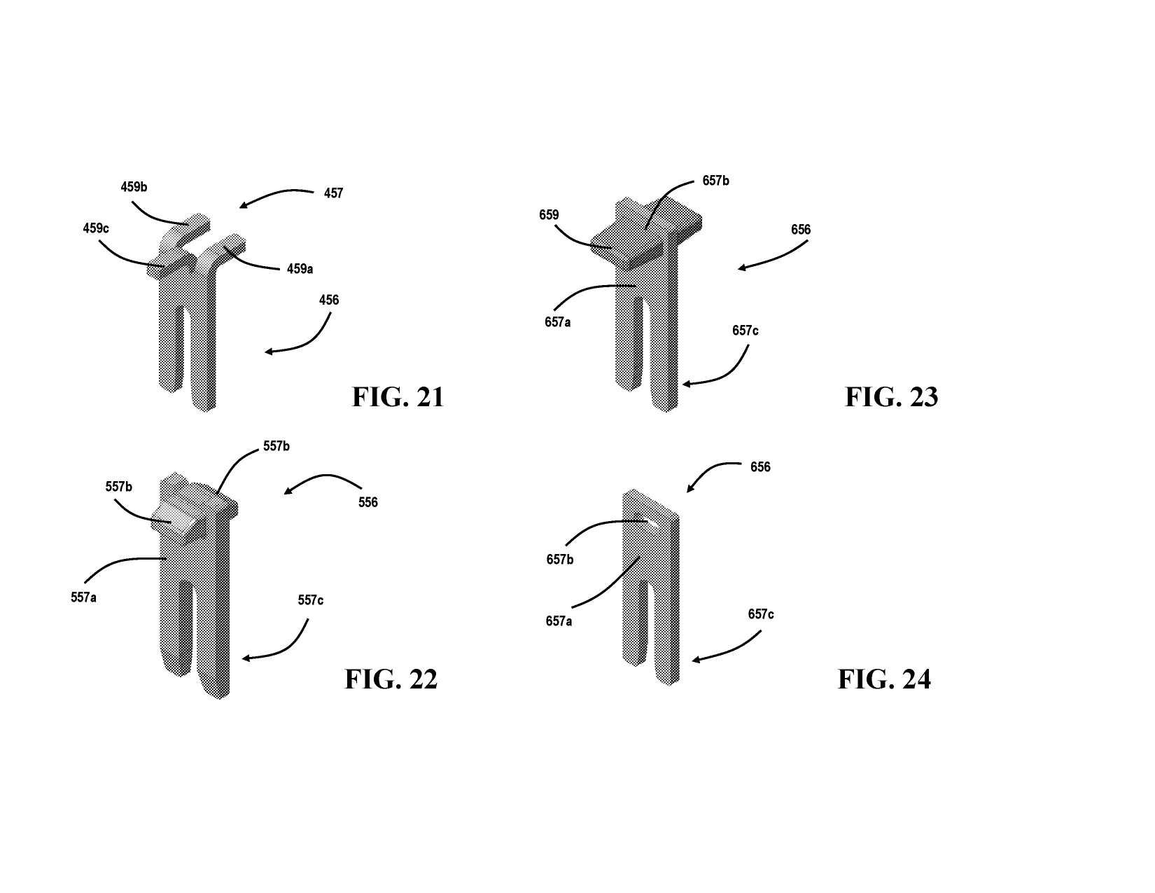

FIG. 21 illustrates folded tab 457 of drive link 456 including a pair of outer fingers 459a, 459b extending in one direction and an inner finger 459c extending in an opposite direction. Fingers 459a, 459b, 459c may extend at angles of, in aspects, about 75 degrees to about 105 degrees; or, in other aspects, about 90 degrees.

Drive links 256, 356, 456 may be formed from metal via stamping, laser cutting, combinations thereof, or in any other suitable manner and/or using any other suitable material(s).

Turning to FIG. 22, another drive link 556 provided in accordance with the present disclosure is shown including a plate body 557a having at least one tab 557b extending transversely from plate body 557a. In aspects, as shown, opposing tabs 557b extend transversely from opposing sides of plate body 557a. Drive link 556 further includes a forked portion 557c disposed at an opposite end of plate body 557a as compared to tab(s) 557b. Drive link 556 may be formed from a plastic material via injection molding, a metal via metal injection molding (MIM), or in any other suitable manner and/or from any other suitable material(s). For example, drive link 556 may be manufacturing via forming liquid metal, powder metal, bulk metallic glass, or other suitable material(s) or configurations thereof to define drive link 556. In aspects, drive link 556 is formed in a one-step process. In other aspects, drive link 556 is formed in a multi-step process, e.g., wherein the plate portion and tab features are created in separate steps. Drive link 556 may otherwise be configured similar to and include any of the features of the drive links detailed above.

Referring to FIGS. 23 and 24, drive link 656 is shown including a plate body 657a defining an opening 657b extending transversely therethrough. Plate body 657a may be formed from metal via stamping, or in any other suitable manner and/or using any other suitable material(s). A tab 659, e.g., formed from plastic, may be overmolded to plate body 657a to extend through opening 657b and transversely from opposing sides of plate body 657a. Drive link 656 further includes a forked portion 657c disposed at an opposite end of plate body 657a as compared to tab 657b. Drive link 556 may otherwise be configured similar to and include any of the features of the drive links detailed above.

While aspects and features of the present disclosure are illustrated and described above as being utilized with a hand-held electrosurgical forceps, it is also contemplated that the aspects and features of the present disclosure be utilized with other surgical instruments and systems, such as robotic surgical systems and what is commonly referred to as “Telesurgery.” Such systems employ various robotic elements to assist the user and allow remote operation (or partial remote operation) of surgical instrumentation. Various robotic arms, gears, cams, pulleys, electric and mechanical motors, etc. may be employed for this purpose and may be designed with a robotic surgical system to assist the user during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to an operating theater or located in a remote location. In this instance, one team of users may prep a patient for surgery and configure the robotic surgical system with one or more of the instruments disclosed herein while another user (or group of user) remotely control the instruments via the robotic surgical system. As can be appreciated, a highly skilled user may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the user to produce a corresponding movement of the working ends of any type of surgical instrument (e.g., end effectors, graspers, knifes, scissors, etc.) which may complement the use of one or more of the aspects described herein. The movement of the master handles may be scaled so that the working ends have a corresponding movement that is different, smaller or larger, than the movement performed by the operating hands of the user. The scale factor or gearing ratio may be adjustable so that the operator can control the resolution of the working ends of the surgical instrument(s).

The master handles may include various sensors to provide feedback to the user relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the user with enhanced tactile feedback simulating actual operating conditions. The master handles may also include a variety of different actuators for delicate tissue manipulation or treatment further enhancing the user’s ability to mimic actual operating conditions.

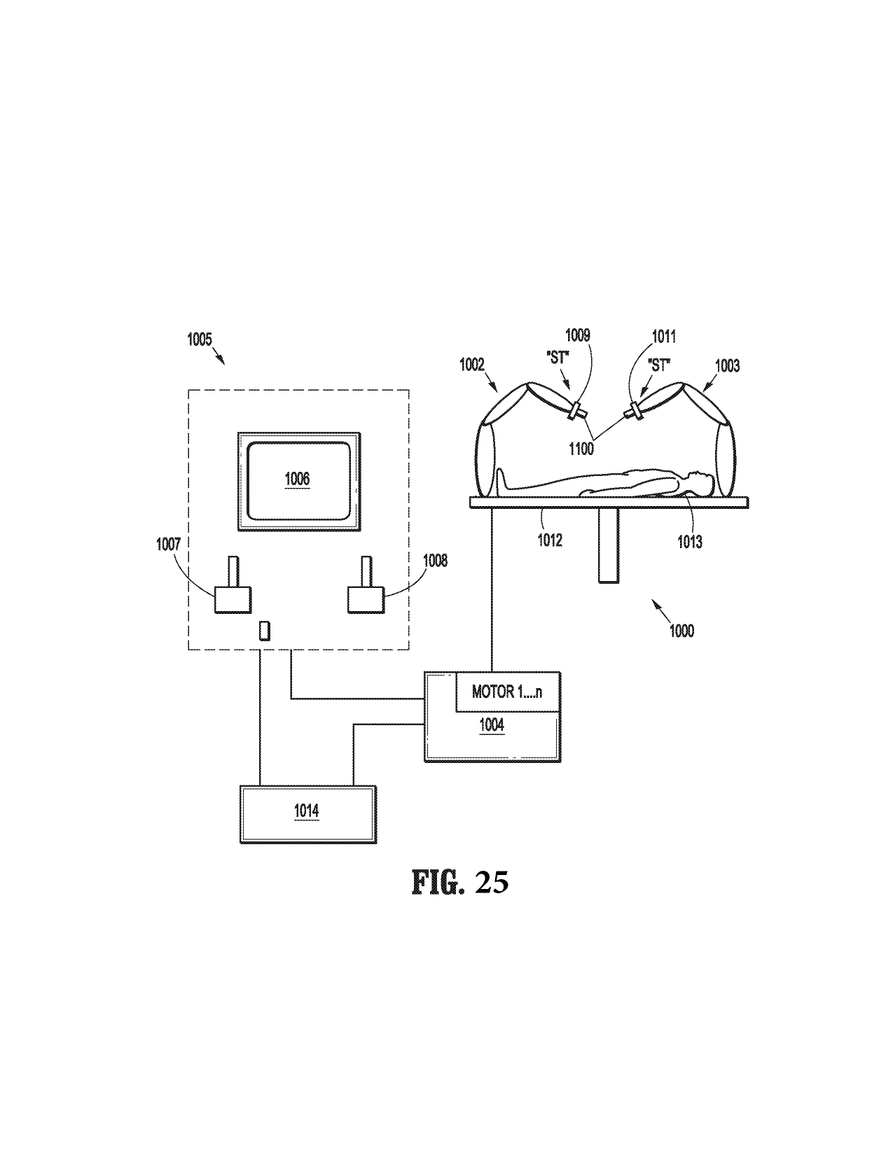

Referring now to FIG. 25, a medical workstation of a robotic surgical system such as detailed above is shown generally as workstation 1000 and generally includes a plurality of robot arms 1002, 1003; a control device 1004; and an operating console 1005 coupled with the control device 1004. The operating console 1005 may include a display device 1006, which may be set up in particular to display three-dimensional images; and manual input devices 1007, 1008, by means of which a user may be able to telemanipulate the robot arms 1002, 1003 in a first operating mode.

Each of the robot arms 1002, 1003 may include a plurality of members, which are connected through joints, and an attaching device 1009, 1011, to which may be attached, for example, a surgical tool “ST” supporting an end effector 1100. The surgical tool “ST” and end effector 1100 may include, for example, the above-detailed features of electrosurgical forceps 10 (FIG. 1) adapted for use in a robotic surgical system. More specifically, the above-detailed handle and trigger mechanisms of electrosurgical forceps 10 may be actuated by robotic actuators, rather than manual actuators, without departing from the present disclosure.

The robot arms 1002, 1003 may be driven by electric drives (not shown) that are connected to the control device 1004. The control device 1004 (e.g., a computer) may be set up to activate the drives, in particular by means of a computer program, in such a way that the robot arms 1002, 1003, their attaching devices 1009, 1011 and thus the surgical tool “ST” (including end effector 1100) execute a desired movement according to a movement defined by means of the manual input devices 1007, 1008. The control device 1004 may also be set up in such a way that it regulates the movement of the robot arms 1002, 1003 and/or of the drives.

The medical workstation 1000 may be configured for use on a patient 1013 lying on a patient table 1012 to be treated in a minimally invasive manner by means of the end effector 1100. The medical workstation 1000 may also include more than two robot arms 1002, 1003, the additional robot arms likewise being connected to the control device 1004 and being telemanipulatable by means of the operating console 1005. A medical instrument or surgical tool (including an end effector 1100) may also be attached to the additional robot arm. Medical workstation 1000 may include a database 1014, in particular coupled to the control device 1004, in which are stored, for example, pre-operative data from the patient/living being 1013 and/or anatomical atlases.

Aspects of this disclosure may be further described by reference to the following examples:

Example 1. A surgical instrument, comprising: a shaft including a slot defined therein and extending longitudinally therealong, the slot defining a width; a drive element slidably disposed within the shaft; a drive link including a first portion positioned externally of the shaft and a second portion extending through the slot into an interior of the shaft to engage the drive element within the shaft, the drive link defining a depth, a width greater than the depth, and a length, wherein the depth of the drive link is less than the width of the slot to enable the drive link to extend through the slot, and wherein the width of the drive link is greater than the width of the slot; and a body disposed about at least a portion of the shaft, wherein the first portion of the drive link is coupled to the body such that translation of the body along the shaft translates the drive element through the shaft.

Example 2. The surgical instrument according to example 1, wherein the length of the drive link is greater than the width of the drive link.

Example 3. The surgical instrument according to example 1, wherein the second portion of the drive link is forked to define first and second spaced-apart prongs configured to engage the drive element therebetween.

Example 4. The surgical instrument according to example 1, where the drive element includes a body and a leg disposed at an angle relative to the body, and wherein the drive link is configured to engage the leg of the drive element.

Example 5. The surgical instrument according to example 1, wherein the drive link defines a plate configuration having opposed faces interconnected by an edge, wherein the length and width of the drive link are defined across the opposed faces and wherein the depth is defined across the edge.

Example 6. The surgical instrument according to example 1, further comprising at least one tab extending transversely from the first portion of the drive link.

Example 7. The surgical instrument according to example 1, wherein the first portion of the drive link is rotatably coupled to the body such the drive link is rotatable independently of the body.

Example 8. A surgical instrument, comprising: a housing; a shaft including a proximal end portion disposed within the housing, the shaft extending distally from the housing, the proximal end portion of the shaft including a slot defined therein and extending longitudinally therealong, the slot defining a width; an end effector assembly disposed at a distal end portion of the shaft; a drive element slidably disposed within the shaft, the drive element configured to actuate a portion of the end effector assembly; and a drive link disposed within the housing and including a first portion positioned externally of the shaft and a second portion extending through the slot into an interior of the shaft to engage the drive element within the shaft, the drive link defining a depth, a width greater than the depth, and a length, wherein the depth of the drive link is less than the width of the slot to enable the drive link to extend through the slot, and wherein the width of the drive link is greater than the width of the slot, wherein translation of the drive link through the slot translates the drive element through the shaft to actuate the portion of the end effector assembly.

Example 9. The surgical instrument according to example 8, wherein the length of the drive link is greater than the width of the drive link.

Example 10. The surgical instrument according to example 8, wherein the second portion of the drive link is forked to define first and second spaced-apart prongs configured to engage the drive element therebetween.

Example 11. The surgical instrument according to example 8, where the drive element includes a body and a leg disposed at an angle relative to the body, and wherein the drive link is configured to engage the leg of the drive element.

Example 12. The surgical instrument according to example 8, wherein the drive link defines a plate configuration having opposed faces interconnected by an edge, wherein the length and width of the drive link are defined across the opposed faces and wherein the depth is defined across the edge.

Example 13. The surgical instrument according to example 8, further comprising a trigger coupled to the housing and the drive link, wherein actuation of the trigger relative to the housing translates the drive link through the slot.

Example 14. The surgical instrument according to example 13, further comprising a body coupling the trigger with the drive link, wherein the first portion of the drive link is captured within the body.

Example 15. A surgical instrument, comprising: an outer shaft; a drive shaft slidably disposed within the outer shaft, the drive shaft including a slot defined therein and extending longitudinally therealong, the slot defining a width; an end effector assembly disposed at a distal end portion of the outer shaft, the end effector assembly including first and second jaw members, at least one of the first or second jaw members movable relative to another of the first or second jaw members to grasp tissue therebetween, the end effector assembly further including a knife movable between the first and second jaw members to cut tissue grasped therebetween; a drive element slidably disposed within the drive shaft, the drive element coupled to move the knife; and

a drive link extending through the slot into an interior of the drive shaft to engage the drive element within the drive shaft, the drive link defining a depth, a width greater than the depth, and a length, wherein the depth of the drive link is less than the width of the slot to enable the drive link to extend through the slot, and wherein the width of the drive link is greater than the width of the slot, wherein translation of the drive link through the slot translates the drive element through the drive shaft to thereby move the knife relative to the first and second jaw members.

Example 16. The surgical instrument according to example 15, wherein the length of the drive link is greater than the width of the drive link.

Example 17. The surgical instrument according to example 15, wherein the outer shaft includes a slot, and wherein the drive link also extends through the slot of the outer shaft.

Example 18. The surgical instrument according to example 15, wherein the drive shaft is slidable through the outer shaft to move the at least one of the first or second jaw members to grasp tissue therebetween, and wherein the drive shaft is configured to apply a force to the at least one of the first or second jaw members of at least 80 lbs.

Example 19. The surgical instrument according to example 15, wherein the drive element includes a body and a leg disposed at an angle relative to the body, and wherein the drive link is configured to engage the leg of the drive element.

Example 20. The surgical instrument according to example 15, wherein the drive link includes a forked portion having first and second spaced-apart prongs configured to engage the drive element therebetween.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. A surgical instrument, comprising:
a shaft including a slot defined therein and extending longitudinally therealong, the slot defining a width;
a drive element slidably disposed within the shaft;
a drive link including a first portion positioned externally of the shaft and a second portion extending through the slot into an interior of the shaft to engage the drive element within the shaft, the drive link defining a depth, a width greater than the depth, and a length, wherein the depth of the drive link is less than the width of the slot to enable the drive link to extend through the slot, and wherein the width of the drive link is greater than the width of the slot; and
a body disposed about at least a portion of the shaft, wherein the first portion of the drive link is coupled to the body such that translation of the body along the shaft translates the drive element through the shaft.

2. The surgical instrument according to claim 1, wherein the length of the drive link is greater than the width of the drive link.

3. The surgical instrument according to claim 1, wherein the second portion of the drive link is forked to define first and second spaced-apart prongs configured to engage the drive element therebetween.

4. The surgical instrument according to claim 1, where the drive element includes a body and a leg disposed at an angle relative to the body, and wherein the drive link is configured to engage the leg of the drive element.

5. The surgical instrument according to claim 1, wherein the drive link defines a plate configuration having opposed faces interconnected by an edge, wherein the length and width of the drive link are defined across the opposed faces and wherein the depth is defined across the edge.

6. The surgical instrument according to claim 1, further comprising at least one tab extending transversely from the first portion of the drive link.

7. The surgical instrument according to claim 1, wherein the first portion of the drive link is rotatably coupled to the body such the drive link is rotatable independently of the body.

8. A surgical instrument, comprising:
a housing;
a shaft including a proximal end portion disposed within the housing, the shaft extending distally from the housing, the proximal end portion of the shaft including a slot defined therein and extending longitudinally therealong, the slot defining a width;
an end effector assembly disposed at a distal end portion of the shaft;
a drive element slidably disposed within the shaft, the drive element configured to actuate a portion of the end effector assembly; and
a drive link disposed within the housing and including a first portion positioned externally of the shaft and a second portion extending through the slot into an interior of the shaft to engage the drive element within the shaft, the drive link defining a depth, a width greater than the depth, and a length, wherein the depth of the drive link is less than the width of the slot to enable the drive link to extend through the slot, and wherein the width of the drive link is greater than the width of the slot,
wherein translation of the drive link through the slot translates the drive element through the shaft to actuate the portion of the end effector assembly.

9. The surgical instrument according to claim 8, wherein the length of the drive link is greater than the width of the drive link.

10. The surgical instrument according to claim 8, wherein the second portion of the drive link is forked to define first and second spaced-apart prongs configured to engage the drive element therebetween.

11. The surgical instrument according to claim 8, where the drive element includes a body and a leg disposed at an angle relative to the body, and wherein the drive link is configured to engage the leg of the drive element.

12. The surgical instrument according to claim 8, wherein the drive link defines a plate configuration having opposed faces interconnected by an edge, wherein the length and the width of the drive link are defined across the opposed faces and wherein the depth is defined across the edge.

13. The surgical instrument according to claim 8, further comprising a trigger coupled to the housing and the drive link, wherein actuation of the trigger relative to the housing translates the drive link through the slot.

14. The surgical instrument according to claim 13, further comprising a body coupling the trigger with the drive link, wherein the first portion of the drive link is captured within the body.

15. A surgical instrument, comprising:
an outer shaft;
a drive shaft slidably disposed within the outer shaft, the drive shaft including a slot defined therein and extending longitudinally therealong, the slot defining a width;
an end effector assembly disposed at a distal end portion of the outer shaft, the end effector assembly including first and second jaw members, at least one of the first or second jaw members movable relative to another of the first or second jaw members to grasp tissue therebetween, the end effector assembly further including a knife movable between the first and second jaw members to cut tissue grasped therebetween;
a drive element slidably disposed within the drive shaft, the drive element coupled to move the knife; and
a drive link extending through the slot into an interior of the drive shaft to engage the drive element within the drive shaft, the drive link defining a depth, a width greater than the depth, and a length, wherein the depth of the drive link is less than the width of the slot to enable the drive link to extend through the slot, and wherein the width of the drive link is greater than the width of the slot,
wherein translation of the drive link through the slot translates the drive element through the drive shaft to thereby move the knife relative to the first and second jaw members.

16. The surgical instrument according to claim 15, wherein the length of the drive link is greater than the width of the drive link.

17. The surgical instrument according to claim 15, wherein the outer shaft includes a slot, and wherein the drive link also extends through the slot of the outer shaft.

18. The surgical instrument according to claim 15, wherein the drive shaft is slidable through the outer shaft to move the at least one of the first or second jaw members to grasp tissue therebetween, and wherein the drive shaft is configured to apply a force to the at least one of the first or second jaw members of at least 80 lbs.

19. The surgical instrument according to claim 15, wherein the drive element includes a body and a leg disposed at an angle relative to the body, and wherein the drive link is configured to engage the leg of the drive element.

20. The surgical instrument according to claim 15, wherein the drive link includes a forked portion having first and second spaced-apart prongs configured to engage the drive element therebetween.
